(19) United States
(12) Patent Application Publication
(10) Pub. No.: US 2010/0010100 A1
(43) Pub. Date: Jan. 14, 2010

(54) DERMATOLOGICAL COMPOSITIONS WITH ANTI-AGING AND SKIN EVEN-TONING PROPERTIES

(76) Inventors: Andrew W. Hinman, San Francisco, CA (US); Guy M. Miller, Monte Sereno, CA (US)

Correspondence Address: MORRISON & FOERSTER LLP, 755 PAGE MILL RD, PALO ALTO, CA, 94304-1018, US

(21) Appl. No.: 12/499,042

(22) Filed: Jul. 7, 2009

Related U.S. Application Data
(60) Provisional application No. 61/134,516, filed on Jul. 9, 2008.

(51) Int. Cl. A61K 31/05; A61K 31/12

(52) U.S. Cl. 514/734; 514/678

(57) ABSTRACT

The present invention relates to methods and compositions comprising benzoquinones such as Duroquinone, for the use of treating, regulating or preventing a skin condition characterized by oxidative stress or a degenerative process. Methods of preventing, lightening or reducing the appearance of visible and/or tactile discontinuities of the skin resulting from skin pigmentation or skin aging are also disclosed.

### CROSS-REFERENCE TO RELATED APPLICATIONS

This application claims priority benefit of U.S. Provisional Patent Application No. 61/134,516, filed Jul. 9, 2008. That application is hereby incorporated by reference herein in its entirety.

### FIELD OF THE INVENTION

The present invention relates to cosmetic and dermatological compositions with anti-aging and skin even-toning properties. The present invention relates to cosmetic and dermatological compositions containing an effective amount of certain benzoquinone or hydroquinone derivatives for improving the appearance of skin affected by visible and/or tactile discontinuities or for delaying the progression of the appearance of visible and/or tactile discontinuities. The present invention relates in particular to cosmetic and dermatological compositions which provide effective protection from damaging oxidation processes in the skin and which provide effective skin anti-aging properties. Furthermore, the present invention relates to cosmetic and dermatological compositions for the prophylaxis and treatment of dermatological skin changes, such as for example skin aging and skin pigmentation, and in particular skin changes produced by oxidative or degenerative processes.

### BACKGROUND OF THE INVENTION

Natural-looking skin is influenced by a number of physiological and genetic factors. Standard definitions of beautiful skin include skin having a transparent quality with uniform undertones of color and no visible or tactile discontinuities. The basis for this natural-looking appearance is in the skin structure itself. The outer layer of human skin is a semi-transparent layer known as the stratum corneum. The transparency of the stratum corneum permits glimpses of the deeper layers of skin, where blood vessels and pigments reside. The pale reddish hue of the blood vessels' hemoglobin, and the brown/black hue of melanin that is the primary skin pigment, combine to produce the skin's color. Ideal skin should also be smooth and even, with no apparent surface flaws in addition to having the transparent look with uniform color distribution.

Skin is composed of a top layer, the epidermis, which is approximately 20 cell layers or about 0.1 mm in thickness, and a lower layer, the dermis, which is from about 1 to about 4 mm in thickness and contains small blood vessels, collagen, elastin and fibroblasts. The dermis provides structural support and nutrients to the epidermis. Aging has been shown to increase cellular heterogeneity of the epidermal layer. Aging does not affect the number of cell layers in the epidermis, but the overall thickness decreases. The supporting dermis is known to thin with age and exposure to the sun and environmental contaminants. The dermal layer provides the support and blood supply for the epidermis, therefore the dermal layer is important in maintaining the elasticity and appearance of the skin. Disruption of the supporting dermis leads directly to sagging and consequent furrowing of the epidermis, i.e., the formation of wrinkles.

Deep wrinkles are also due to continual stretching and contraction of both the dermis and epidermis. Currently, these deep wrinkles or furrows may only be eliminated by plastic surgery or by collagen injections directly beneath the depressed areas. The fine wrinkles that occur with age and prolonged exposure to the sun and other environmental contaminants are the direct result of deterioration of the supporting dermal layer.

As a result of the aging process and damage caused by incident radiation, a disruption of the collagen bundles that provide support to the epidermis is observed. Collagen exists normally in dense, organized patterns. During the aging process collagen becomes disorganized and less supportive of the epidermis and the dermis loses elasticity. There is also progressive loss of circulatory support from the small blood vessels that are more numerous and close to the surface in young skin. The result of aging on skin is a deterioration of the dermal layer, i.e. fewer fibroblasts, less collagen, less elastin and less circulatory support. Consequently, the normal stretching and contraction of the skin leads to damage of the dermis that is not readily corrected, and wrinkling results.

Additionally, with age, the epidermis thins, sebaceous secretions decrease, and the skin becomes more susceptible to dryness, chapping, and fissuring. This results in wrinkling and other forms of unevenness, including, but not limited to, increased pore size, flaking, mottling, discoloration, age spots and skin lines.

Dermatologists and cosmetologists have directed their efforts to improving the appearance of skin using agents known to stimulate the growth and proliferation of epidermal cells. Newly proliferated cells provide more structure and hold more moisture, giving the skin a younger appearance. One method of causing new skin cell proliferation is accomplished by use of an irritant or chemical peel in which the uppermost layers of the epidermis are caused to slough off, leading to proliferation and replacement with new epidermal cells. While such treatment is recognized to provide some cosmetic improvement, it does not address the major causative factor, namely, the compromised supporting dermal layer.

Considerable effort has also been expended to find ways to prevent adverse changes in the skin brought about by ultraviolet (UV) exposure. Preventative approaches include physically blocking or absorbing the UV radiation before it can enter the skin using UV absorbing compounds. Skin problems in aging individuals can result from a variety of extrinsic or intrinsic factors such as harmful UV radiation from the sun, exposure to the environment, stress, fatigue, disease, or a combination thereof.

Many people at different stages of their life are concerned with the degree of pigmentation of their skin and may wish to reduce the skin darkening, or may wish to lighten or even-tone their natural skin color. The mechanism by which skin pigmentation is formed, melanogenesis, is particularly complex and schematically involves the following main steps: Tyrosine→L-Dopa→Dopaquinone→Dopachrome→Melanins. The first two reactions in this series are catalyzed by the enzyme tyrosinase. The activity of tyrosinase is promoted by the action of α-melanocyte stimulating hormone or UV rays. It is well established that a substance has a depigmenting effect if it acts directly on the vitality of the epidermal melanocytes where melanogenesis normally occurs and/or if it interferes with one of the stages in melanin biosynthesis. Pigmentation disorders can take a variety of forms like hyperpigmentation, hypopigmentation, and uneven pigmentation, and include but are not limited to melasma (mask of pregnancy or chloasma), liver spots (which often develop with age) and leukoderma such as vitiligo. Some of the pigmentation occurs as a side effect of birth control pills, as a result of skin damage such as a persistent result of acne, burns, bites and other skin injuries, as after-burn scars, as cicatrical spots, as stretch mark scars, and as dark circles and puffiness under and around the eyes. The degree of pigmentation disorders of the skin in many cases increases with the age of the individuals.

The present invention relates to methods for reducing or improving the appearance of visible and/or tactile discontinuities in skin associated with aging, age-related damage, or damage resulting from harmful ultraviolet radiation, such as that contained in sunlight, pollution and other environmental insults, stress, or fatigue. The present invention also relates to methods for reducing the appearance of coloration due to pigmentation disorders. The invention concerns compositions and methods of improving skin appearance by lifting and firming the skin. The invention concerns compositions and methods of improving skin appearance by alleviating skin deterioration associated with age and reducing the appearance of coloration due to pigmentation disorders simultaneously. The present invention concerns compositions comprising certain quinone derivatives for reducing or preventing the appearance of skin pigmentation and the skin problems arising with age. The present invention relates to methods for reducing or improving the appearance of visible and/or tactile discontinuities in skin associated with inflammation.

In the United States, the most commonly used treatment for hyperpigmentation is 1,4-benzenediol, which is known as hydroquinone. Treatment with hydroquinone interferes with the action of tyrosinase, which is an enzyme used in the synthesis of melanin, and compositions are sold across the counter at about 2% hydroquinone and by prescription at higher concentrations. Hydroquinone compositions are effective but have some undesirable side effects. These can be burning, redness, sensitization and irritation in some patients. U.S. Pat. No. 4,526,179 refers to certain hydroquinone fatty esters that have good activity and are less irritating and more stable than hydroquinone. Japanese Patent Application No. 27909/86 (JP 61-27909) refers to other hydroquinone derivatives that do not have the drawbacks of hydroquinone but that have relatively poor efficacy. Other compounds with a hydroquinone core structure have been described in the patent literature, for example, U.S. Pat. No. 5,449,518 refers to 2,5-dihydroxyphenyl carboxylic acid derivatives, and European Patent Application EP 341,664A1 and PCT International Publication WO 99/15148 refer to certain resorcinol derivatives as tyrosinase inhibitors.

A variety of additional agents have been applied to the skin to lighten the skin. Such agents include but are not limited to kojic acid, licorice and its derivatives, ascorbic acid and its derivatives, arbutin, bearberry, Glycyrrhiza glabra and its derivatives, Chlorella vulgaris extract, perilla extract, and coconut fruit extract. Perilla extract is disclosed as a whitening agent in U.S. Pat. No. 5,980,904 and Japanese Publications Nos. 07-025742, 07-187989, 10-265322, 2001-163759 and 2001-181173. Coconut fruit extract is disclosed as a whitening agent in Japanese Patent No. 2896815 B2. These agents only address sub-optimally the lightening of the skin without addressing the effects of aging on the skin.

It would be desirable to have a safe and non-toxic composition for the treatment or prevention of the pigmentation disorders. It would also be desirable to have a composition with anti-aging benefits for the skin. It would be even more desirable to have a treatment or prevention of pigmentation disorders with anti-aging benefits for the skin in one composition or in a combination of two compositions. Compositions comprising quinones of the present invention fill this need.

### DISCLOSURE OF THE INVENTION

The quinone derivatives of this invention, which are defined below and used in the various methods of this invention, are useful in the treatment or prevention of one or more dermatological conditions for which the subject being treated desires, for medicinal or cosmetic purposes, to prevent, lighten, reduce or treat the signs or appearance of skin aging and/or pigmentation of skin affected by the one or more conditions.

The invention thus provides a cosmetic or dermatological composition comprising one or more benzoquinones of formula I or their reduced form of formula Ia:

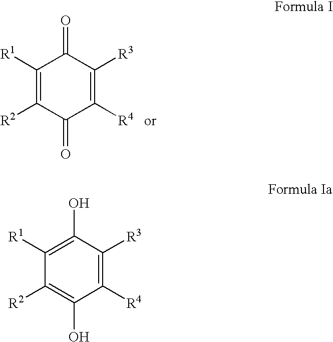

wherein R¹, R², R³, and R⁴ are independently selected from (C₁-C₆)alkyl, (C₁-C₆) arylalkyl or aryl, where the aryl group can be further substituted with (C₁-C₆)alkyl, (C₁-C₆)alkoxy, hydroxy, halogen or (C₁-C₆)haloalkyl,
and mixtures thereof.

In one embodiment R¹, R², R³, and R⁴ are independently selected from (C₁-C₄)alkyl.

In another embodiment, R¹, R², R³, and R⁴ are methyl, and the compound is Duroquinone (also named 2,3,5,6-tetramethylcyclohexa-2,5-diene-1,4-dione) of Formula Ib, its reduced form 2,3,5,6-tetramethylbenzene-1,4-diol of Formula Ic, or mixtures thereof.

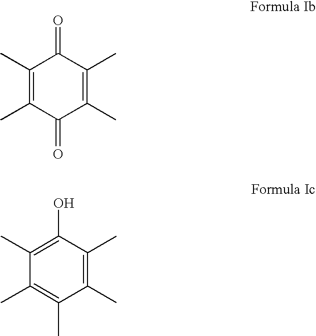

In another embodiment, the composition comprises Duroquinone of formula Ib. In yet another embodiment, the composition comprises the compound of formula Ic. In yet another embodiment, the composition comprises a mixture of compound of formula Ib and of compound of formula Ic.

In another embodiment, the composition comprises a compound of formula I wherein R¹, R², and R³ are methyl and R⁴ is unsubstituted phenyl; this subset of formula I is designated as formula Iaa. When in reduced (hydroquinone) form, this subset is designated as Iaa-R.

In another embodiment, the composition comprises a compound of formula I wherein R¹, R², and R³ are methyl and R⁴ is phenyl substituted with halogen or (C₁-C₆)haloalkyl; this subset of compounds of formula I is designated as compounds of formula Iab. When in reduced (hydroquinone) form, this subset is designated as Iab-R.

In another embodiment, the composition comprises a compound of formula I wherein R¹, R², and R³ are methyl and R⁴ is phenyl substituted with Cl, Br, F or I; this subset of compounds of formula I is designated as compounds of formula Iac. When in reduced (hydroquinone) form, this subset is designated as Iac-R. In some exemplary embodiments R¹, R², and R³ are methyl and R⁴ is p-chloro-phenyl, and the compound is 3-(4-chlorophenyl)-2,5,6-trimethylcyclohexa-2,5-diene-1,4-dione, or, in reduced form, 3-(4-chlorophenyl)-2,5,6-trimethylbenzene-1,4-diol.

In another embodiment, the composition comprises a compound of formula I wherein R¹, R², and R³ are methyl and R⁴ is phenyl substituted with CF₃; this subset of compounds of formula I is designated as compounds of formula Iad. When in reduced (hydroquinone) form, this subset is designated as Iad-R. In some exemplary embodiments, the compound is 2,5,6-trimethyl-3-(4-(trifluoromethyl)-phenyl)cyclohexa-2,5-diene-1,4-dione, or, in reduced form, 2,5,6-trimethyl-3-(4-(trifluoromethyl)-phenyl)benzene-1,4-diol.

In another embodiment, the composition comprises a compound of formula I wherein R¹, R², and R³ are methyl and R⁴ is (C₁-C₄)phenylalkyl where the phenyl group is unsubstituted; this subset of compounds of formula I is designated as compounds of formula Iae. When in reduced (hydroquinone) form, this subset is designated as Iae-R. In an exemplary embodiment R¹, R², and R³ are methyl and R⁴ is benzyl or phenylethyl, and the compounds are 3-benzyl-2,5,6-trimethylcyclohexa-2,5-diene-1,4-dione or 2,5,6-trimethyl-3-phenethylcyclohexa-2,5-diene-1,4-dione, or, in reduced form, 3-benzyl-2,5,6-trimethyl benzene-1,4-diol or 2,5,6-trimethyl-3-phenethyl benzene-1,4-diol.

In another embodiment, the composition comprises a compound of formula I wherein R¹, R², and R³ are methyl and R⁴ is (C₁-C₄)phenylalkyl where the phenyl group is substituted with halogen or (C₁-C₄)haloalkyl; this subset of compounds of formula I is designated as compounds of formula Iaf. When in reduced (hydroquinone) form, this subset is designated as Iaf-R.

In another embodiment, the composition comprises any of the compounds of formula Iaa, Iab, Iac, Iad, Iae, or Iaf, or any of the specific embodiments thereof, in their reduced hydroquinone form; these compounds are designated as compounds of formula Iaa-R, Iab-R, Iac-R, Iad-R, Iae-R, or Iaf-R.

In one embodiment, the present invention relates to cosmetic or dermatological compositions comprising one or more compounds of formula I, formula Ia, formula Iaa, formula Iaa-R, formula Iab, formula Iab-R, formula Iac, formula Iac-R, formula Iad, formula Iad-R, formula Iae, formula Iae-R, formula Iaf, formula Iaf-R, formula Ib, or formula Ic, having effective protection from damaging oxidation or degenerative processes. In some embodiments, the compositions of the present invention reduce or treat or prevent the signs of skin aging or reduce or retard the appearance of signs of skin aging. In other embodiments, the compositions of the present invention reduce or treat or prevent pigmentation disorders and uneven tone of the skin.

In another embodiment, the present invention relates to cosmetic and dermatological compositions comprising one or more compounds of formula I, formula Ia, formula Iaa, formula Iaa-R, formula Iab, formula Iab-R, formula Iac, formula Iac-R, formula Iad, formula Iad-R, formula Iae, formula Iae-R, formula Iaf, formula Iaf-R, formula Ib, or formula Ic, for the prophylaxis and treatment of cosmetic and dermatological skin changes, for example signs of skin aging and skin pigmentation. In some embodiments, the skin changes are produced by oxidative or degenerative processes.

In another embodiment, the present invention relates to cosmetic and dermatological compositions comprising one or more compounds of formula I, formula Ia, formula Iaa, formula Iaa-R, formula Iab, formula Iab-R, formula Iac, formula Iac-R, formula Iad, formula Iad-R, formula Iae, formula Iae-R, formula Iaf, formula Iaf-R, formula Ib, or formula Ic, for the prophylaxis and treatment of cosmetic and dermatological skin changes concurrently with providing effective skin anti-aging properties. In some embodiments, the cosmetic and dermatological skin changes are associated with visible and/or tactile discontinuities of the skin.

In some embodiments, the present invention relates to cosmetic and dermatological compositions comprising one or more compounds of formula I, formula Ia, formula Iaa, formula Iaa-R, formula Iab, formula Iab-R, formula Iac, formula Iac-R, formula Iad, formula Iad-R, formula Iae, formula Iae-R, formula Iaf, formula Iaf-R, formula Ib, or formula Ic, for preventing, lightening or reducing visible signs from sun aging.

In another embodiment, the present invention relates to cosmetic and dermatological compositions comprising one or more compounds of formula I, formula Ia, formula Iaa, formula Iaa-R, formula Iab, formula Iab-R, formula Iac, formula Iac-R, formula Iad, formula Iad-R, formula Iae, formula Iae-R, formula Iaf, formula Iaf-R, formula Ib, or formula Ic, for the prophylaxis or treatment of dermatological conditions comprising unevenness or pigmentation of the skin.

In another embodiment, the present invention relates to cosmetic and dermatological compositions comprising one or more compounds of formula I, formula Ia, formula Iaa, formula Iaa-R, formula Iab, formula Iab-R, formula Iac, formula Iac-R, formula Iad, formula Iad-R, formula Iae, formula Iae-R, formula Iaf, formula Iaf-R, formula Ib, or formula Ic, for reducing the appearance of visible and/or tactile discontinuities in skin associated with aging, age-related damage, or damage resulting from harmful factors, such as those contained in sunlight, harmful ultraviolet radiation, pollution and other environmental insults, stress, or fatigue.

In another embodiment, the present invention relates to cosmetic and dermatological compositions comprising one or more compounds of formula I, formula Ia, formula Iaa, formula Iaa-R, formula Iab, formula Iab-R, formula Iac, formula Iac-R, formula Iad, formula Iad-R, formula Iae, formula Iae-R, formula Iaf, formula Iaf-R, formula Ib, or formula Ic, for preventing, lightening or reducing the appearance of visible and/or tactile discontinuities of the skin such as increased pore size, flaking, mottling, wrinkles, furrows, and skin lines.

In other embodiments, the present invention relates to cosmetic and dermatological compositions comprising one or more compounds of formula I, formula Ia, formula Iaa, formula Iaa-R, formula Iab, formula Iab-R, formula Iac, formula Iac-R, formula Iad, formula Iad-R, formula Iae, formula Iae-R, formula Iaf, formula Iaf-R, formula Ib, or formula Ic, for preventing, lightening or reducing the appearance of visible discontinuities of the skin resulting from the aging processes.

In another embodiment, the present invention relates to cosmetic and dermatological compositions comprising one or more compounds of formula I, formula Ia, formula Iaa, formula Iaa-R, formula Iab, formula Iab-R, formula Iac, formula Iac-R, formula Iad, formula Iad-R, formula Iae, formula Iae-R, formula Iaf, formula Iaf-R, formula Ib, or formula Ic, for preventing, lightening or reducing the appearance of visible discontinuities of the skin such as pigmentation, age spots, vitiligo and melasma.

In some embodiments, the present invention relates to cosmetic and dermatological compositions comprising one or more compounds of formula I, formula Ia, formula Iaa, formula Iaa-R, formula Iab, formula Iab-R, formula Iac, formula Iac-R, formula Iad, formula Iad-R, formula Iae, formula Iae-R, formula Iaf, formula Iaf-R, formula Ib, or formula Ic, for preventing, lightening or reducing the appearance of visible discontinuities of the skin such as pigmentation resulting from extrinsic insults such as harmful factors contained in sunlight, harmful ultraviolet radiation, pollution and other environmental insults, stress and fatigue.

In some embodiments, the present invention relates to cosmetic and dermatological compositions comprising one or more compounds of formula I, formula Ia, formula Iaa, formula Iaa-R, formula Iab, formula Iab-R, formula Iac, formula Iac-R, formula Iad, formula Iad-R, formula Iae, formula Iae-R, formula Iaf, formula Iaf-R, formula Ib, or formula Ic, for preventing, lightening or reducing the appearance of dark circles, dark spots and uneven skin tone.

In another embodiment, the present invention relates to cosmetic and dermatological compositions comprising one or more compounds of formula I, formula Ia, formula Iaa, formula Iaa-R, formula Iab, formula Iab-R, formula Iac, formula Iac-R, formula Iad, formula Iad-R, formula Iae, formula Iae-R, formula Iaf, formula Iaf-R, formula Ib, or formula Ic, for reducing the appearance of visible and/or tactile discontinuities in skin associated with inflammation. In some embodiments, the visible discontinuities are caused by post-inflammatory hypopigmentation. In other embodiments, the inflammation is caused by rosacea. In other embodiments, the inflammation is caused by diaper rash. In other embodiments, the inflammation is caused by acne. In other embodiments, the inflammation is caused by dermatitis such as atopic dermatitis, contact dermatitis, or seborrheic dermatitis. In other embodiments, the inflammation is caused by poison ivy or poison oak. In other embodiments, the inflammation is caused by erythema. In other embodiments, the inflammation is caused by psoriasis.

The present invention does not relate to the treatment or prevention of conditions caused, exacerbated or transmitted by bacterial, in particular staphylococcal or propionibacterial, activity.

In another embodiment, the invention relates to cosmetic, dermatological and topical pharmaceutical compositions comprising one or more compounds of formula I, formula Ia, formula Iaa, formula Iaa-R, formula Iab, formula Iab-R, formula Iac, formula Iac-R, formula Iad, formula Iad-R, formula Iae, formula Iae-R, formula Iaf, formula Iaf-R, formula Ib, or formula Ic for reducing skin inflammation.

The invention further provides a cosmetic composition comprising a topical cosmetically acceptable or dermatologically acceptable carrier in combination with any one or more of the compounds of formula I, formula Ia, formula Iaa, formula Iaa-R, formula Iab, formula Iab-R, formula Iac, formula Iac-R, formula Iad, formula Iad-R, formula Iae, formula Iae-R, formula Iaf, formula Iaf-R, formula Ib, or formula Ic.

The invention further provides a cosmetic composition comprising a pharmaceutically acceptable carrier in combination with any one or more of the compounds of formula I, formula Ia, formula Iaa, formula Iaa-R, formula Iab, formula Iab-R, formula Iac, formula Iac-R, formula Iad, formula Iad-R, formula Iae, formula Iae-R, formula Iaf, formula Iaf-R, formula Ib, or formula Ic.

In one embodiment, the present invention relates to methods for reducing the appearance of visible and/or tactile discontinuities in skin with a composition comprising one or more compounds of formula I, formula Ia, formula Iaa, formula Iaa-R, formula Iab, formula Iab-R, formula Iac, formula Iac-R, formula Iad, formula Iad-R, formula Iae, formula Iae-R, formula Iaf, formula Iaf-R, formula Ib, or formula Ic, wherein the composition is included in a topical formulation.

In one embodiment, the present invention relates to methods for reducing the appearance of visible and/or tactile discontinuities in skin with a composition comprising one or more compounds of formula I, formula Ia, formula Iaa, formula Iaa-R, formula Iab, formula Iab-R, formula Iac, formula Iac-R, formula Iad, formula Iad-R, formula Iae, formula Iae-R, formula Iaf, formula Iaf-R, formula Ib, or formula Ic, wherein the composition is included in a topical pharmaceutical formulation.

In one embodiment, the present invention relates to methods for reducing the appearance of visible and/or tactile discontinuities in skin with a composition comprising one or more compounds of formula I, formula Ia, formula Iaa, formula Iaa-R, formula Iab, formula Iab-R, formula Iac, formula Iac-R, formula Iad, formula Iad-R, formula Iae, formula Iae-R, formula Iaf, formula Iaf-R, formula Ib, or formula Ic, wherein the composition is formulated for transdermal administration.

The present invention further provides a method of lightening skin in a human while providing reduction or treatment or prevention of signs of skin aging, comprising administering to said human an even-toning, skin-lightening or pigmentation-reducing effective amount of one or more compounds of formula I, formula Ia, formula Iaa, formula Iaa-R, formula Iab, formula Iab-R, formula Iac, formula Iac-R, formula Iad, formula Iad-R, formula Iae, formula Iae-R, formula Iaf, formula Iaf-R, formula Ib, or formula Ic. In a particular embodiment, the present invention provides a method of lightening skin in a human in need of said treatment while providing reduction or treatment or prevention of signs of skin aging, comprising administering to said human a skin-lightening effective amount of a composition comprising a compound of formula I, formula Ia, formula Ib or formula Ic.

The present invention further provides a method of improving the appearance of skin in a human, comprising administering to said human a wrinkle-reducing effective amount of a composition comprising a compound of formula I, formula Ia, formula Iaa, formula Iaa-R, formula Iab, formula Iab-R, formula Iac, formula Iac-R, formula Iad, formula Iad-R, formula Iae, formula Iae-R, formula Iaf, formula Iaf-R, formula Ib, or formula Ic. In a particular embodiment, the present invention provides a method of reducing the appearance of wrinkles, fine lines and furrows in a human in need of said treatment, comprising administering to said human an effective amount of a composition comprising a compound of formula I, formula Ia, formula Iaa, formula Iaa-R, formula Iab, formula Iab-R, formula Iac, formula Iac-R, formula Iad, formula Iad-R, formula Iae, formula Iae-R, formula Iaf, formula Iaf-R, formula Ib, or formula Ic.

The present invention further provides a method of improving the appearance of skin in a human, comprising administering to said human a pigmentation-reducing effective amount of a composition comprising a compound of formula I, formula Ia, formula Iaa, formula Iaa-R, formula Iab, formula Iab-R, formula Iac, formula Iac-R, formula Iad, formula Iad-R, formula Iae, formula Iae-R, formula Iaf, formula Iaf-R, formula Ib, or formula Ic. In a particular embodiment, the present invention provides a method of reducing the appearance of pigmentation in a human in need of said treatment, comprising administering to said human an effective amount of a composition comprising a compound of formula I, formula Ia, formula Iaa, formula Iaa-R, formula Iab, formula Iab-R, formula Iac, formula Iac-R, formula Iad, formula Iad-R, formula Iae, formula Iae-R, formula Iaf, formula Iaf-R, formula Ib, or formula Ic. In some embodiment, the invention provides a method of treating pigmentation or reducing the appearance of pigmentation or prophylaxis against the appearance of pigmentation. In some embodiments the patient has a pigmentation disorder selected from age spots, vitiligo and melasma.

In another embodiment, the invention relates to a method of regulating skin condition characterized by oxidative stress comprising administering to a subject exhibiting said skin condition a composition comprising one or more compounds of formula I, formula Ia, formula Iaa, formula Iaa-R, formula Iab, formula Iab-R, formula Iac, formula Iac-R, formula Iad, formula Iad-R, formula Iae, formula Iae-R, formula Iaf, formula Iaf-R, formula Ib, or formula Ic.

In another embodiment, the invention relates to a method of regulating and/or preventing signs of skin aging comprising administering to a subject exhibiting skin damage due to aging, a composition comprising one or more compounds of formula I, formula Ia, formula Iaa, formula Iaa-R, formula Iab, formula Iab-R, formula Iac, formula Iac-R, formula Iad, formula Iad-R, formula Iae, formula Iae-R, formula Iaf, formula Iaf-R, formula Ib, or formula Ic.

In another embodiment, the invention relates to a method of regulating and/or preventing signs of skin damage due to extrinsic factors comprising administering to a subject exhibiting skin damage a composition comprising one or more compounds of formula I, formula Ia, formula Iaa, formula Iaa-R, formula Iab, formula Iab-R, formula Iac, formula Iac-R, formula Iad, formula Iad-R, formula Iae, formula Iae-R, formula Iaf, formula Iaf-R, formula Ib, or formula Ic. In some embodiments, the extrinsic factors lead to diaper rash, erythema, UV radiation damage, sunburn, photoaging, contact dermatitis, and combinations thereof.

The present invention further provides a method of reducing the appearance of pigmentation and aging processes in the skin in a human, comprising administering to said human an effective amount of a composition comprising one or more compounds of formula I, formula Ia, formula Iaa, formula Iaa-R, formula Iab, formula Iab-R, formula Iac, formula Iac-R, formula Iad, formula Iad-R, formula Iae, formula Iae-R, formula Iaf, formula Iaf-R, formula Ib, or formula Ic in combination with another therapeutic agent. In one embodiment, the present invention provides a method of reducing the appearance of pigmentation and aging processes in the skin in a human, comprising administering to said human an effective amount of a composition comprising one or more compounds of formula I, formula Ia, formula Iaa, formula Iaa-R, formula Iab, formula Iab-R, formula Iac, formula Iac-R, formula Iad, formula Iad-R, formula Iae, formula Iae-R, formula Iaf, formula Iaf-R, formula Ib, or formula Ic in combination with an antioxidant. In one embodiment, the present invention provides a method of reducing the appearance of pigmentation and aging processes in a human in need of said treatment, comprising administering to said human an effective amount of a composition comprising one or more compounds of formula I, formula Ia, formula Iaa, formula Iaa-R, formula Iab, formula Iab-R, formula Iac, formula Iac-R, formula Iad, formula Iad-R, formula Iae, formula Iae-R, formula Iaf, formula Iaf-R, formula Ib, or formula Ic in combination with ascorbic acid or derivatives thereof. In another embodiment, the present invention provides a method of reducing the appearance of pigmentation and aging processes in a human in need of said treatment, comprising administering to said human an effective amount of a composition comprising one or more compounds of formula I, formula Ia, formula Iaa, formula Iaa-R, formula Iab, formula Iab-R, formula Iac, formula Iac-R, formula Iad, formula Iad-R, formula Iae, formula Iae-R, formula Iaf, formula Iaf-R, formula Ib, or formula Ic in combination with alpha-tocopherol or any mixture of tocopherols or derivatives thereof. In another embodiment, the present invention provides a method of reducing the appearance of pigmentation and aging processes in a human in need of said treatment, comprising administering to said human an effective amount of a composition comprising one or more compounds of formula I, formula Ia, formula Iaa, formula Iaa-R, formula Iab, formula Iab-R, formula Iac, formula Iac-R, formula Iad, formula Iad-R, formula Iae, formula Iae-R, formula Iaf, formula Iaf-R, formula Ib, or formula Ic in combination with tocotrienols or any mixture of tocopherols and tocotrienols or derivatives thereof. In yet another embodiment, the present invention provides a method of reducing the appearance of pigmentation and aging processes in a human in need of said treatment, comprising administering to said human an effective amount of a composition comprising one or more compounds of formula I, formula Ia, formula Ib or formula Ic in combination with ascorbic acid and alpha-tocopherol or derivatives thereof. In other embodiments, the present invention provides a method of reducing the appearance of pigmentation and aging processes in a human in need of said treatment, comprising administering to said human an effective amount of a composition comprising one or more compounds of formula I, formula Ia, formula Iaa, formula Iaa-R, formula Iab, formula Iab-R, formula Iac, formula Iac-R, formula Iad, formula Iad-R, formula Iae, formula Iae-R, formula Iaf, formula Iaf-R, formula Ib, or formula Ic in combination with retinoids or an exfoliating agent. When administered in combination, the therapeutic agents can be formulated as separate compositions that are given at the same time or different times, or the therapeutic agents can be given as a single composition.

The present invention further embraces a product comprising instructions directing a user to apply a composition including a skin care composition comprising one or more compounds of formula I, formula Ia, formula Iaa, formula Iaa-R, formula Iab, formula Iab-R, formula Iac, formula Iac-R, formula Iad, formula Iad-R, formula Iae, formula Iae-R, formula Iaf, formula Iaf-R, formula Ib, or formula Ic.

The present invention further provides a kit, comprising a container comprising one or more specific compounds or dermatological compositions of the present invention that lighten skin pigmentation. The kit may further comprise printed instructions as a label or a package insert directing the use of the enclosed compound or composition to lighten skin pigmentation.

The present invention further provides a kit, comprising a container comprising one or more specific compounds or dermatological compositions of the present invention that reduce or treat or prevent the signs of skin aging. The kit may further comprise printed instructions as a label or a package insert directing the use of the enclosed compound or composition to reduce or treat or prevent the signs of skin aging.

The present invention further provides a kit, comprising a container comprising one or more specific compounds or dermatological compositions of the present invention that lighten skin while concurrently reducing or treating or preventing the signs of skin aging. The kit may further comprise printed instructions as a label or package insert directing the use of the enclosed compound or composition to lighten skin pigmentation while concurrently reducing or treating or preventing the signs of skin aging.

The present invention further embraces a method of promoting a product by directing a user to apply a skin care composition comprising a skin care composition of any of the foregoing embodiments.

The present invention further embraces the use of a composition of any of the foregoing embodiments in the manufacture of a cosmetic or dermatological composition for treating a mammalian subject of a dermatologic condition to prevent, regulate or treat signs of skin aging or skin pigmentation, or to reduce the appearance of skin aging or skin pigmentation.

### METHODS OF CARRYING OUT THE INVENTION

### Definitions

As used herein, the terms “even-toning”, “whitening ”, “lightening” and “depigmentation” agent are used interchangeably throughout this document. For purposes of skin lightening, topical application of skin lightening agent should have a lightening effect on only the area to be treated, preferably produce no or minimal irritation nor post-inflammatory secondary pigmentation, nor cause an allergic reaction. In addition, the skin lightening should be effective for normal cutaneous pigmentation and its excesses, including, but not limited to, lentigo senilis, chloasma, cicatrical brown spots, and hyperpigmentation after use of photosensitizing products. Preferably, the skin lightening should be effective while simultaneously providing anti-aging skin benefits.

As used herein, a “skin-lightening or pigmentation reducing amount of a compound of formula I, formula Ia, formula Iaa, formula Iaa-R, formula Iab, formula Iab-R, formula Iac, formula Iac-R, formula Iad, formula Iad-R, formula Iae, formula Iae-R, formula Iaf, formula Iaf-R, formula Ib, or formula Ic,”, means an amount or concentration of the compound capable of detectably lightening skin or reducing pigmentation in a human, as determined by any standard assay. The active compound is typically administered in a dermatological or pharmaceutical composition for a standard course of treatment that produces the desired result of skin depigmentation.

As used herein, “administering to skin in need of such treatment” means contacting (e.g., by use of the hands or an applicator such as, but not limited to, a wipe, tube, roller, spray, or patch) the area of skin in need such treatment or an area of skin proximate to the area of skin in need of such treatment.

As used herein, “composition” means a composition suitable for topical administration to the skin.

As used herein, the term “cosmetics” includes make-up, foundation, and skin care products. The term “make-up” refers to products that leave color on the face, including foundations, blacks and browns, e.g., mascara, concealers, eye liners, brow colors, eye shadows, blushers, lip colors, and so forth. The term “foundation” refers to liquid, creme, mousse, pancake, compact, concealer, or like products that even out the overall coloring of the skin. Foundation is typically manufactured to work better over moisturized and/or oiled skin. The term “skin care products” refers to products used to treat or otherwise care for, moisturize, improve, or clean the skin. Products contemplated by the phrase “skin care products” include, but are not limited to, adhesives, bandages, anhydrous occlusive moisturizers, antiperspirants, facial wash cleaners, cold cream, deodorants, soaps, occlusive drug delivery patches, powders, tissues, wipes, solid emulsion compact, anhydrous hair conditioners, medicated shampoos, scalp treatments and the like.

As used herein, the term “cosmetically-acceptable” or “dermatologically-acceptable” means that the compositions or components thereof so-described are suitable for use in contact with human skin without undue toxicity, incompatibility, instability, irritation, allergic response, or the like.

As used herein, the term “cosmetically acceptable carrier”, “cosmetically acceptable excipient”, “dermatologically acceptable carrier” or “dermatologically acceptable excipient” includes any and all solvents, dispersion media, coatings, antibacterial and antifungal agents, isotonic and absorption delaying agents and the like, that are cosmetically acceptable or dermatologically acceptable. The use of such media and agents for cosmetically active substances is well known in the art. Except insofar as any conventional media or agent is incompatible with the active ingredient, its use in the cosmetic compositions is contemplated. Supplementary active ingredients can also be incorporated into the compositions. Dermatologically acceptable carriers are suitable for topical application to the skin, have good aesthetic properties, are compatible with the active agents of the present invention and any other components, and will not cause any safety or toxicity concerns. A safe and effective amount of carrier is from about 50% to about 99.99% or about 50% to about 99%, preferably from about 80% to about 99.9% or about 75% to about 99%, more preferably from about 90% to about 98%, and most preferably from about 90% to about 95% or about 85% to about 95% of the composition. The percentages are preferably percent by weight.

As used herein, the term “effective amount” refers to that amount of a compound of the present invention that is sufficient to effect treatment, as defined below, when administered to a subject in need of such treatment. The effective amount will vary depending upon the subject and disease condition being treated and the like, all of which can readily be determined by one of ordinary skill in the art.

As used herein, “regulating skin condition” includes regulating the appearance of a skin condition, including visible and/or tactile discontinuities in skin such as, but not limited to, skin wrinkles, elasticity or the sagging of skin, oily skin, puffiness of skin under the eyes, striae, and stretch marks. Regulating skin condition includes improving skin appearance and/or feel. Regulating skin condition includes lifting and improving the tone and firmness of the skin. Regulation skin condition includes even-toning the skin and reducing pigmentation.

As used herein, “regulating the signs of skin aging” includes cosmetically regulating the appearance of one or more of such signs of skin aging as defined herein.

As used herein, “signs of skin aging” include, but are not limited to, all outward visibly and tactilely perceptible manifestations as well as any other macro or micro effects due to skin aging. Such signs may be induced or caused by intrinsic factors or extrinsic factors, e.g., chronological aging and/or environmental damage (e.g., sunlight, UV, smoke, ozone, pollutants, stress, etc.). These signs may result from processes which include, but are not limited to, the development of textural discontinuities such as wrinkles, including both fine superficial wrinkles and coarse deep wrinkles, skin lines, facial frown lines, expression lines, rhytides, dermatoheliosis, dark circles under the eyes, photo damage, premature skin aging, crevices, bumps, pits, large pores (e.g., associated with adnexal structures such as sweat gland ducts, sebaceous glands, or hair follicles), “orange-peel” skin appearance, dryness, scaliness, flakiness and/or other forms of skin unevenness or roughness; abnormal desquamation (or exfoliation) or abnormal epidermal differentiation (e.g., abnormal skin turnover) such as scaliness, flakiness, keratoses, hyperkeratinization; inadequate skin moisturization (or hydration) such as caused by skin barrier damage or environmental dryness; loss of skin elasticity (loss and/or inactivation of functional skin elastin) such as elastosis, sagging (including puffiness and dark circles in the eye area and jowls), loss of skin firmness, loss of skin tightness, loss of skin recoil from deformation; loss of muscle tone, non-melanin skin discoloration such as under-eye circles, blotching (e.g., uneven red coloration due to, e.g., rosacea), sallowness (pale color), discoloration caused by telangiectasia or spider vessels; melanin-related hyperpigmented (or unevenly pigmented) skin regions such as age spots (liver spots, brown spots) and freckles; post-inflammatory hyperpigmentation such as that which occurs following an inflammatory event (e.g., as an acne lesion, in-grown hair, insect/spider bite or sting, scratch, cut, wound, abrasion, and the like); atrophy such as, but not limited to, that associated with aging or steroid use; other histological or microscopic alterations in skin components such as ground substance (e.g., hyaluronic acid, glycosaminoglycans, etc.), collagen breakdown and structural alterations or abnormalities (e.g., changes in the stratum corneum, dermis, epidermis, the skin vascular system such as telangiectasia or spider vessels); tissue responses to insult such as itch or pruritus; and alterations to underlying tissues (e.g., subcutaneous fat, cellulite, muscles, septae, and the like), especially those proximate to the skin.

As used herein, the terms “skin condition”, “dermatologic condition”, and “dermatological condition” are used interchangeably.

As used herein, the term “sunscreen” may include, but is not limited to, organic or inorganic sunscreens, such as methoxycinnamate, oxybenzone, avobenzone, and the like; sun blocks such as titanium oxide and zinc oxide; and skin protectants or mixtures thereof.

As used herein, the term “topical application” means to apply or spread the compositions of the present invention onto the surface of the skin.

As used herein, the terms “treat” and “treating”, and the like refer to reversing, alleviating, or inhibiting the progress of, the disorder or condition to which such term applies, or one or more symptoms of such disorder or condition. The term “treatment”, as used herein, refers to the act of treating, as “treating” is defined immediately above. The term “treatment” or “treating” includes the reduction in appearance of skin imperfections irrelevant of the mechanism of action. One of ordinary skill in the art will appreciate that the endpoint of treatment chosen in a particular case will vary according to the disease, condition, or disorder being treated, the outcome desired by the patient, subject, or treating physician, and other factors. Where the composition is being used to lighten skin color such as, for example, to reverse hyperpigmentation caused by, for example, diseases such as melasma or age spots, any one of a number of endpoints can be chosen. For example, endpoints can be defined subjectively such as, for example, when the subject is simply “satisfied” with the results of the treatment. For pharmacological compositions, the endpoint can be determined by the patient's, or the treating physician's, satisfaction with the results of the treatment. Alternatively, endpoints can be defined objectively. For example, the patient's or subject's skin in the treated area can be compared to a color chart. Treatment is terminated when the color of the skin in the treated area is similar in appearance to a color on the chart. Alternatively, the reflectance of the treated skin can be measured, and treatment can be terminated when the treated skin attains a specified reflectance. Alternatively, the melanin content of the treated skin can be measured. Treatment can be terminated when the melanin content of the treated skin reaches a specified value. Melanin content can be determined in any way known to the art, including by histological methods, with or without enhancement by stains for melanin.

As used herein, the term “tocopherols or tocotrienols” encompasses a family of molecules characterized by a 6-chromanol ring structure and a side chain at the 2-position. Tocopherols possess a 4′,8′,12′-trimethyltridecyl phytol side chain, while tocotrienols possess an unsaturated phytol side chain. As used herein, the term tocopherol or tocotrienols means alpha-, beta-, gamma- or delta-, epsilon- and zeta-tocopherol or tocotrienols (see The Merck Index (1996), Merck & Co., Whitehouse Station, N.J. 1620-1621 and 1712, and references cited therein), as well as Vitamin E. The term tocopherol also includes cosmetically acceptable esters, for example tocopherol acetate, tocopherol lineate, or tocopherol stearate. The term tocopherol also includes mixtures of tocopherols, tocotrienols and/or stereoisomers as well as enriched compositions comprising at least 50% of any tocopherol or tocotrienol. The tocopherols and tocotrienols can be of natural or synthetic origin.

As used herein, the term “retinoids” means retinol, retinal, retinyl palmitate, retinyl linoleate, retinoic acid or esters, as well as synthetic or natural Vitamin A. The term “retinol” includes the following isomers of retinol: all-trans-retinol, 13-cis-retinol, 11-cis-retinol, 9-cis-retinol, 3,4-didehydro-retinol. Retinyl ester is an ester of retinol, as defined above. Retinyl esters suitable for use in the present invention are C₁-C₃₀ esters of retinol, preferably C₂-C₂₀ esters, and most preferably C₂-C₃ and C₁₆ esters because they are more commonly available. Some esters for use in the present invention may be selected from retinyl palmitate, retinyl acetate, retinyl propionate and retinyl linoleate. Retinoyl ester is an ester of retinoic acid with an alcohol. Retinoyl esters suitable for use in the present invention include C₁-C₃₀ alcohol esters of retinoic acid, preferably C₂-C₂₀ esters and most preferably C₂-C₃ and C₁₆ esters. Some retinoyl esters for use in the present invention comprise the linoleyl alcohol ester of retinoic acid, the hexanedecanol ester of retinoic acid, the oleic alcohol ester of retinoic acid, retinoyl ascorbate, and the linolenyl alcohol ester of retinoic acid.

As used herein, the term “(C₁-C₆)-alkyl” is intended to embrace a saturated linear, branched, cyclic, or a combination of linear and/or branched and/or cyclic hydrocarbon chain and/or ring of 1 to 6 carbon atoms. Examples of “(C₁-C₆)-alkyl” are methyl, ethyl, n-propyl, isopropyl, cyclopropyl, n-butyl, isobutyl, sec-butyl, t-butyl, cyclobutyl, cyclopropyl-methyl, methyl-cyclopropyl, pentyl where the point of attachment of the pentyl group to the remainder of the molecule can be at any location on the pentyl fragment, cyclopentyl, hexyl where the point of attachment of the hexyl group to the remainder of the molecule can be at any location on the hexyl fragment, and cyclohexyl. This term includes mono and divalent hydrocarbon chains, i.e. (C₁-C₆)-alkyl and (C₁-C₆)-alkylene chains of 1 to 6 carbon atoms.

As used herein, the term “(C₁-C₆)-haloalkyl” is intended to embrace any (C₁-C₆)-alkyl substituent having at least one halogen substituent; the halogen can be attached via any valence on the (C₁-C₆)-alkyl group. One subset of (C₁-C₆)-haloalkyl is —CF₃, —CCl₃, —CBr₃, and —CI₃. Another subset of (C₁-C₆)-haloalkyl is —CHF₂, —CHCl₂, —CHBr₂, and —CHI₂. Another subset of (C₁-C₆)-haloalkyl is —CH₂F, —CH₂Cl, —CH₂Br, and —CH₂I. Another subset of (C₁-C₆)-haloalkyl is the subset of (C₁-C₆)-perhaloalkyls where all available valences are replaced by halogens. Another subset of (C₁-C₆)-haloalkyl is the subset of (C₁-C₆)-perfluoroalkyl, where all available valences are replaced by fluorines. Another subset of (C₁-C₆)-haloalkyl is the subset of (C₁-C₆)-perchloroalkyl, that is, (C₁-C₆)-alkyl with all available valences replaced by chlorines.

As used herein, the term “(C₁-C₆)-alkoxy” refers to an alkyl group of 1 to 6 carbon atoms linked via an oxygen atom to the remainder of the molecule. Examples of alkoxy groups include, but are not limited to, groups such as methoxy (—OCH₃), ethoxy (—OCH₂CH₃), propyloxy(propoxy) (either n-propoxy (—OCH₂CH₂CH₃) or i-propoxy(-OCH(CH₃)₂), and butoxy (either n-butoxy, i-butoxy, sec-butoxy, or tert-butoxy).

As used herein, the term “aryl” is intended to embrace an aromatic cyclic hydrocarbon group of from 6 to 20 carbon atoms having a single ring (e.g., phenyl) or multiple condensed (fused) rings (e.g., naphthyl or anthryl).

### Methods of the Invention

The compounds of this invention can be mixed as cosmetics, cosmeceuticals, quasi-drugs (where applicable), or pharmaceutical drugs. The compounds of this invention can appropriately be mixed with other components. Examples of such components include oily components such as hydrocarbons, fats and oils such as liquid paraffin, squalene, vaseline, cetyl alcohol, isostearyl alcohol, cetyl-2-ethylhexanoate, 2-octyldodecyl alcohol, glycerin, glycerin triisostearate, nut oils, and lanolin, as well as wax, silicone, surfactants, thickeners, neutralizers, antiseptics, germicides, anti-oxidants, powder components, pigments, perfumes, ultraviolet light absorbents, drugs, metallic sealant, and pH modifiers.

Occurrences in the skin of noticeable but undesired pigmentation as a result of overproduction or underproduction of melanin or of noticeable uneven texture as a result of aging can be reduced, treated or prevented using the methods of the present invention. Cosmetic applications for methods of the present invention include the topical application of compositions containing one or more of the compounds of the present invention to enhance or otherwise alter the visual appearance of skin. The cosmetic compositions of the present invention are also useful to provide a smoother or softer skin appearance.

The active compounds used in this invention can also be used in combination with skin peeling agents (including glycolic acid or trichloroacetic acid face peels) or skin exfoliating agents (including retinoids, such as retinoic acid or retinol) to lighten skin tone and prevent repigmentation. The appropriate dose regimen, the amount of each dose administered, and specific intervals between doses of the active compound will depend upon the particular active compound employed, the condition of the patient being treated, and the nature and severity of the disorder or condition being treated. Preferably, the active compound is administered in an amount and at an interval that results in the desired treatment of or improvement in the disorder or condition being treated.

An active compound used in this invention can also be used in combination with sun screens (UVA or UVB blockers) to prevent repigmentation; to protect against sun or UV-induced skin darkening or to enhance their ability to reduce skin melanin and their skin bleaching action. An active compound used in this invention can also be used in combination with any compounds that interact with retinoic acid receptors and accelerate or enhance the invention's ability to reduce skin melanin and skin bleaching action, or enhance the invention's ability to prevent the accumulation of skin melanin. An active compound used in this invention can also be used in combination with 4-hydroxyanisole. An active compound used in this invention can also be used in combination with ascorbic acid, its derivatives and ascorbic-acid based products (such as magnesium ascorbate) or other products with an anti-oxidant mechanism (such as resveratrol, tocopherols, tocotrienols and derivatives) which accelerate or enhance their ability to reduce skin melanin and their skin bleaching action.

In some embodiments of the present invention, the composition further comprises a soybean extract that is a blend of compounds isolated from soybean. The soybean extract may contain only a portion of the soybean (e.g., an extract of the soybean such as a lipid reduced soybean powder or filtered soymilk) or may contain the entire soybean (e.g., a ground powder of the soybean). The soybean extract may be in the form of a fluid (e.g., soymilk) or a solid (e.g., a soybean powder or soymilk powder).

As one skilled in the art would know in view of this disclosure, an active compound used in the methods of the present invention may be used alone or in combination with other compounds known in the art to affect melanin synthesis, particularly other melanin synthesis inhibitors, including tyrosinase inhibitors. Such inhibitors include those currently known in the art and those to be developed in the future. Known inhibitors include various resorcinol derivatives, kojic acid derivatives, hydroquinone, melamine, and various types of plant extracts, among others. For example, any of the compounds used according to a skin-lightening method of the present invention may be used in combination with a tyrosinase inhibitor or other skin-whitening agent, including any one or more of those agents, including compounds or extracts, described in the following patent publications: U.S. Pat. No. 4,278,656 to Nagai et al, describing the use of kojic acid and its ester derivatives; U.S. Pat. No. 4,959,393 to Torihara et al., describing the use of resorcinol derivatives; U.S. Pat. No. 5,164,182 to Luanratana Omboon describing the use of Mulberry extracts, U.S. Pat. No. 5,580,549 to Fukuda et al. describing the use of various hydroxybenzoic acid derivatives; U.S. Pat. No. 5,723,109 to L'Oreal describing the use of salicylic acid derivatives; U.S. Pat. No. 6,123,959 to Jones et al., describing the use of liposomes containing combinations of competitive inhibitors, such as arbutin, and non-competitive inhibitors, such as aloesin, of melanin synthesis; U.S. Pat. No. 6,132,740 to Hu, describing the use of various resorcinol derivatives; U.S. Pat. No. 6,159,482 to Tuloup et al., describing the use of various hydroxyphenyl oxamate derivatives; U.S. Pat. No. 6,365,135 to L'Oreal, describing the use of various phenolic amides; U.S. Pat. No. 6,514,538 to Shiseido Co. Ltd., describing the use of Withania plant extracts; U.S. Pat. Publ, No. 2006188559 to Neis describing a combination using alpha arbutin and bearberry extract; WO 99/64025 by Fytokem Prod. Inc., describing the use of various dicotyledonous plant extracts; U.S. Pat. No. 6,348,204 by L'Oreal, describing the use of combinations of mulberry and skullcap extracts with salicylic acid derivatives; WO 00/56702 by Pfizer Inc., describing various resorcinol derivatives; JP 5221846 by Kunimasa Tomoji, describing the use of kojic acid amino acid or peptide derivatives; JP 7242687 by Shiseido Co. Ltd., published Sep. 19, 1995, describing the use of Trichoderma extracts; JP 7324023 by Itogawa H, published Dec. 12, 1995, describing the use of Pseudostellariae radix extracts; JP 8012552 by Shiseido Co. Ltd., describing the use of Amor seco extracts; JP 8012554 by Shiseido Co. Ltd., describing the use of Jabonciilo extracts; JP 8012557 by Shiseido Co. Ltd., describing the use of Huaca extracts; JP 8012560 by Shiseido Co. Ltd., describing the use of Copaiba extracts; JP 8012561 by Shiseido Co. Ltd., describing the use of Arnica extracts; JP 8134090 by Fujisawa, describing the use of galactosyl-kojic acid derivatives; JP 8277225 by Kansai Koso K K, describing the use of Autocarpus incisus extracts; JP 9002967 by Sanki Shoji K K, describing the use of Prunus domestica LINN extracts; JP 9295927 by Yagi Akira, describing the use of Aloe vera extracts; JP 10072330 by Kansai Kouso, describing the use of resveratrol derivatives; JP 10081626 by Kamiyama K K, published Mar. 31, 1998, describing the use of 4-substituted benzoic acids; JP 10101543 by Kansai Kouso K K, describing the use of certain flavonoids; JP 11071231 by Maruzen Pharm., describing the use of bakuchiol; JP 11079934 by Kyodo Nyugyo, published Mar. 23, 1999, describing the use of low molecular weight thiol from sake lees; JP 11246347 by Shiseido Co. Ltd., describing the use of Achillea millefolium extracts; JP 11246344 by Shiseido Co. Ltd., describing the use of Gliricidia extracts; JP 2000-080023 by Kanebo Ltd., published Mar. 21, 2000, describing the use of metallothionine inducers; JP 2000-095663 by Kose K K, describing the use of various plant extracts; JP 2000-159681 by Hai Tai Confectionery Co. Ltd., describing the use of grape seed extract; JP-7206753 by Nikken Food K K, describing the use of dihydroxycurcumin derivatives; JP-59157009 by Yakurigaku Chuou K E. describing the use of beta-thujaplicin, hydroquinone or a pyrone compound in combination with a melanin adsorbent; JP 2001019618, by Shiseido describing the use of jurubidine or isojurubidine; JP 2002029959 by Shiseido describing the use of extracts of Rosa centifolia L. or Rosa gallica L.; JP 2004315534 by Access Business Group Int Llc describing the use of Asparagus officinalis, Cimicifuga racemosa L. or a mixture thereof; JP 2005041821 by Shiseido describing the use of extracts of Garcinia plant; JP 2007063224 by Kobayashi Pharma describing the use of biotins; JP 2007091635 by Maruzen Pharma describing the use of Phaseolus atropurpureus; JP 2008013481 by Univ. of Tokushima describing the use of extracts from Alpinia speciosa; KR 20040078449 by Enbioeng Co Ltd. describing the use of extracts of berberin baicalinate; TW 281863 by Taiyen Biotech Co Ltd, describing the use extracts of extracts of leaves of Podocarpus; and CN 101102746 by Young Chung Se, describing the use of extracts of Vaccinium uliginosum; among others; which patent publications are incorporated herein by reference in their entireties.

This invention also relates to methods of lightening or reducing the pigmentation of skin and/or of reducing uneven texture in which an active compound used in this invention, and one or more of the other active ingredients, such as those referred to above, are administered together as part of the same pharmaceutical composition, as well as methods in which they are administered separately as part of an appropriate dose regimen designed to obtain the benefits of the combination therapy. The appropriate dose regimen, the amount of each dose administered, and specific intervals between doses of each active agent will depend upon the specific combination of active agents employed, the condition of the patient being treated, and the nature and severity of the disorder or condition being treated. Such additional active ingredients will generally be administered in amounts less than or equal to those for which they are effective as single topical therapeutic agents.

An active compound of this invention will generally be administered in the form of a dermatological or cosmetic composition comprising the compound of formula I, formula Ia, formula Iaa, formula Iaa-R, formula Iab, formula Iab-R, formula Iac, formula Iac-R, formula Iad, formula Iad-R, formula Iae, formula Iae-R, formula Iaf, formula Iaf-R, formula Ib or formula Ic, together with a dermatologically acceptable carrier or solvent. Alternatively, an active compound of this invention can be administered in the form of a pharmaceutical composition comprising the compound of formula I, formula Ia, formula Iaa, formula Iaa-R, formula Iab, formula Iab-R, formula Iac, formula Iac-R, formula Iad, formula Iad-R, formula Iae, formula Iae-R, formula Iaf, formula Iaf-R, formula Ib or formula Ic, together with a pharmaceutically acceptable carrier or solvent.

In the depigmenting compositions according to the present invention, the concentration of the active compound of the invention is generally between 0.01 and 10%, for example between 0.1 and 5%, relative to the total weight of the composition.

The compositions of the present invention can be applied directly to the skin. Alternatively, they can be delivered by various transdermal drug delivery systems, such as transdermal patches as known in the art. For example, for topical administration, the active ingredient can be formulated in a solution, gel, lotion, ointment, cream, suspension, paste, liniment, powder, tincture, aerosol, patch, or the like in a pharmaceutically or cosmetically acceptable form by methods well known in the art. The composition can be any of a variety of forms common in the pharmaceutical or cosmetic arts for topical application to animals or humans, including solutions, lotions, sprays, creams, ointments, salves, gels, etc., as described below. Exemplary agents are those that are viscous enough to remain on the treated area, those that do not readily evaporate, and/or those that are easily removed by rinsing with water, optionally with the aid of soaps, cleansers and/or shampoos. Actual methods for preparing topical formulations are known or apparent to those skilled in the art, and are described in detail in Remington's Pharmaceutical Sciences, (1990); and Pharmaceutical Dosage Forms and Drug Delivery Systems, 6th ed., Williams & Wilkins (1995).

The compositions may be made into a wide variety of product types that include but are not limited to solutions, suspensions, lotions, creams, gels, toners, sticks, sprays, ointments, cleansing liquid washes and solid bars, shampoos and hair conditioners, pastes, foams, powders, mousses, shaving creams, wipes, strips, patches, electrically-powered patches, wound dressing and adhesive bandages, hydrogels, film-forming products, facial and skin masks, make-up such as foundations, eye liners, and eye shadows, and the like. These product types may contain several types of cosmetically-acceptable carriers including, but not limited to solutions, suspensions, emulsions such as microemulsions and nanoemulsions, gels, solids and liposomes.

The compositions can be formulated as solutions. Solutions typically include an aqueous or organic solvent (e.g., from about 50% to about 99.99% or from about 90% to about 99% of a cosmetically acceptable aqueous or organic solvent). Examples of suitable organic solvents include: propylene glycol, polyethylene glycol (200-600), polypropylene glycol (425-2025), glycerol, 1,2,4-butanetriol, sorbitol esters, 1,2,6-hexanetriol, ethanol, and mixtures thereof. One example of such solvents is a mixture of ethanol/polyethylene glycol (80/20).

A lotion can be made from such a solution. Lotions typically contain from about 1% to about 20% (e.g., from about 5% to about 10%) of an emollient(s) and from about 50% to about 90% (e.g., from about 60% to about 80%) of water.

Another type of product that may be formulated from a solution is a cream. A cream typically contains from about 5% to about 50% (e.g., from about 10% to about 20%) of an emollient(s) and from about 45% to about 85% (e.g., from about 50% to about 75%) of water.

Yet another type of product that may be formulated from a solution is an ointment. An ointment may contain a simple base of animal, vegetable, or synthetic oils or semi-solid hydrocarbons. An ointment may contain from about 2% to about 10% of an emollient(s) plus from about 0.1% to about 2% of a thickening agent(s). Examples of thickening agents include, but are not limited to, those set forth in the ICI Handbook (International Cosmetic Ingredient Dictionary and Handbook) pp. 1693-1697.

The compositions useful in the present invention can also be formulated as emulsions. If the carrier is an emulsion, from about 1% to about 10% (e.g., from about 2% to about 5%) of the carrier contains an emulsifier(s). Emulsifiers may be nonionic, anionic or cationic. Examples of emulsifiers include, but are not limited to, those set forth in the ICI Handbook, pp. 1673-1686.

Lotions and creams can be formulated as emulsions. Typically such lotions contain from 0.5% to about 5% of an emulsifier(s), while such creams would typically contain from about 1% to about 20% (e.g., from about 5% to about 10%) of an emollient(s); from about 20% to about 80% (e.g., from 30% to about 70%) of water; and from about 1% to about 10% (e.g., from about 2% to about 5%) of an emulsifier(s).

Single emulsion skin care preparations, such as lotions and creams, of the oil-in-water type and water-in-oil type are well-known in the art and are useful in the subject invention. Multiphase emulsion compositions, such as the water-in-oil-in-water type or the oil-in-water-in-oil type, are also useful in the subject invention. In general, such single or multiphase emulsions contain water, emollients, and emulsifiers as essential ingredients.

The compositions of this invention can also be formulated as a gel (e.g., an aqueous, alcohol, alcohol/water, or oil gel using a suitable gelling agent(s)). Suitable gelling agents for aqueous and/or alcoholic gels include, but are not limited to, natural gums, acrylic acid and acrylate polymers and copolymers, and cellulose derivatives (e.g., hydroxymethyl cellulose and hydroxypropyl cellulose). Suitable gelling agents for oils (such as mineral oil) include, but are not limited to, hydrogenated butylene/ethylene/styrene copolymer and hydrogenated ethylene/propylene/styrene copolymer. Such gels typically contains between about 0.1% and 5%, by weight, of such gelling agents.

In order to enhance the percutaneous absorption of the active ingredients, one or more of a number of agents can be added in the topical formulations including, but not limited to, dimethylsulfoxide, dimethylacetamide, dimethylformamide, surfactants, azone, alcohol, acetone, propylene glycol and polyethylene glycol. In addition, physical methods can also be used to enhance transdermal penetration such as, e.g., by iontophoresis or sonophoresis. Alternatively, or in addition, liposomes may be employed.

A topically applied composition of the invention contains a pharmaceutically effective agent that lightens skin as described herein, and those ingredients as are necessary for use as a carrier, such as an emulsion, a cream, an ointment, an aqueous solution, a lotion or an aerosol. Non-limiting examples of such carriers may be found in U.S. Pat. No. 5,691,380 to Mason et al., issued Nov. 25, 1997; and U.S. Pat. No. 5,968,528 to Deckner et al., issued Oct. 19, 1999; which are incorporated herein by reference. Suitable pharmaceutical carriers are further described in Remington's Pharmaceutical Sciences, 17th ed., Mack Publishing Company, Easton, Pa. (1990).

The carrier utilized in the compositions of the invention can be in a wide variety of forms. These include emulsion carriers, including, but not limited to, oil-in-water, water-in-oil, water-in-oil-in-water, and oil-in-water-in-silicone emulsions, a cream, an ointment, an aqueous solution, a lotion or an aerosol. As will be understood by the skilled artisan, a given component will distribute primarily into either the water or oil/silicone phase, depending on the water solubility/dispersibility of the component in the composition.

Dermatological formulations of the present invention may typically comprise a derivative of any compound or composition of the present invention and optionally, a polar solvent. Solvents suitable for use in the formulations of the present invention include any polar solvent capable of dissolving the derivative of the invention. Suitable polar solvents may include: water; alcohols (such as ethanol, propyl alcohol, isopropyl alcohol, hexanol, and benzyl alcohol); polyols (such as propylene glycol, polypropylene glycol, butylene glycol, hexylene glycol, maltitol, sorbitol, and glycerine); and panthenol dissolved in glycerine, flavor oils and mixtures thereof. Mixtures of these solvents can also be used. Exemplary polar solvents may be polyhydric alcohols and water. Examples of solvents may include glycerine, panthenol in glycerine, glycols such as propylene glycol and butylene glycol, polyethylene glycols, water and mixtures thereof. Additional polar solvents for use may be alcohols, glycerine, panthenol, propylene glycol, butylene glycol, hexylene glycol and mixtures thereof.

An emollient may also be added to the cosmetic/dermatological compositions of the present invention. The emollient component can comprise fats, oils, fatty alcohols, fatty acids and esters which aid application and adhesion, yield gloss and most importantly provide occlusive moisturization. Suitable emollients for use may be isostearic acid derivatives, isopropyl palmitate, lanolin oil, diisopropyl dimerate, maleated soybean oil, octyl palmitate, isopropyl isostearate, cetyl lactate, cetyl ricinoleate, tocopheryl acetate, acetylated lanolin alcohol, cetyl acetate, phenyl trimethicone, glyceryl oleate, tocopheryl linoleate, wheat germ glycerides, arachidyl propionate, myristyl lactate, decyl oleate, propylene glycol ricinoleate, isopropyl linoleate, pentaerythrityl tetrastearate, neopentylglycol dicaprylate/dicaprate, hydrogenated coco-glycerides, isononyl isononanoate, isotridecyl isononanoate, myristyl myristate, triisocetyl citrate, cetyl alcohol, octyl dodecanol, oleyl alcohol, panthenol, lanolin alcohol, linoleic acid, linolenic acid, sucrose esters of fatty acids, octyl hydroxystearate and mixtures thereof. Examples of other suitable emollients can be found in the Cosmetic Bench Reference, pp. 1.19-1.22 (1996), or in the International Cosmetic Ingredient Dictionary and Handbook, eds. Wenninger and McEwen, pp. 1656-61, 1626, and 1654-55 (The Cosmetic, Toiletry, and Fragrance Assoc., Washington, D.C., 7.sup.th Edition, 1997) (hereinafter “ICI Handbook”), incorporated herein by reference. Suitable emollients may include polar emollient emulsifiers (such as linear or branched chained polyglycerol esters) and non-polar emollients. The emollient component typically may comprise from about 1% to about 90%, preferably from about 10% to about 80%, more preferably from about 20% to about 70%, and most preferably from about 40% to about 60%, of the cosmetic composition.

By “polar emollient,” as used herein, is meant any emollient emulsifier having at least one polar moiety and wherein the solubility (at 30° C.) of the cytoprotective derivative compound in the polar emollient is greater than about 1.5%, preferably greater than about 2%, more preferably greater than about 3%. Suitable polar emollients may include, but are not limited to, polyol ester and polyol ethers such as linear or branched chained polyglycerol esters and polyglycerol ethers. Non-limiting examples of such emollients may include PG3 diisostearate, polyglyceryl-2-sesquiisostearate, polyglyceryl-5-distearate, polyglyceryl-10-distearate, polyglyceryl-10-diisostearate, acetylated monoglycerides, glycerol esters, glycerol tricaprylate/caprate, glyceryl ricinoleate, glyceryl isostearate, glyceryl myristate, glyceryl linoleate, polyalkylene glycols such as PEG 600, monoglycerides, 2-monolaurin, sorbitan esters and mixtures thereof.

By “non-polar emollient,” as used herein, means any emollient emulsifier possessing no or minimal permanent electric moments. Suitable non-polar emollients may include, but are not limited to, esters and linear or branched chained hydrocarbons. Non-limiting examples of such emollients may include isononyl isononanoate, isopropyl isostearate, octyl hydroxystearate, diisopropyl dimerate, lanolin oil, octyl palmitate, isopropyl palmitate, paraffins, isoparaffins, acetylated lanolin, sucrose fatty acid esters, isopropyl myristate, isopropyl stearate, mineral oil, silicone oils, dimethicone, allantoin, isohexadecane, isododecane, petrolatum, and mixtures thereof. The solubility of the compound in polar or non-polar emollients may be determined according to methods known in the art.

Suitable oils include esters, triglycerides, hydrocarbons and silicones. These can be a single material or a mixture of one or more materials. They may normally comprise from 0% to about 100%, preferably from about 5% to about 90%, and most preferably from about 70% to about 90% of the emollient component.

Oils that act as emollients also impart viscosity, tackiness, and drag properties to cosmetic compositions such as lipstick. Examples of suitable oils may include acrylic triglycerides; caprice triglyceride; isostearyl triglyceride; atopic triglyceride; propylene glycol myristyl acetate; lanolin; lanolin oil; polybutene; isopropyl palmitate; isopropyl myristate; isopropyl isostearate; diethyl sebacate; diisopropyl adipate; tocopheryl acetate; tocopheryl linoleate; hexadecyl stearate; ethyl lactate; cetyl oleate; cetyl ricinoleate; oleyl alcohol; hexadecyl alcohol; octyl hyroxystearate; octyl dodecanol; wheat germ oil; hydrogenated vegetable oils; castor oil; petrolatum; modified lanolins; branched-chain hydrocarbons; alcohols and esters; corn oil; cottonseed oil; olive oil; palm kernel oil; rapeseed oil; safflower oil; jojoba oil; evening primrose oil; avocado oil, mineral oil, shea butter, octylpalmitate, maleated soybean oil, glycerol trioctanoate, diisopropyl dimerate, and volatile and non-volatile silicone oils including phenyl trimethicone.

Suitable oils for use herein may be acetylglycerides, octanoates, and decanoates of alcohols and polyalcohols, such as those of glycol and glycerol, the ricinoleates of alcohols and polyalcohols such as cetyl ricinoleate, PG-3 diisostearate, polyglycerol ethers, polyglyerol esters, caprylic triglycerides, capric triglycerides, isostearic triglyceride, adipic triglyceride, phenyl trimethicone, lanolin oil, polybutene, isopropyl palmitate, isopropyl isostearate, cetyl ricinoleate, octyl dodecanol, oleyl alcohol, hydrogenated vegetable oils, castor oil, modified lanolins, octyl palmitate, lanolin oil, maleated soybean oil, cetyl ricinoleate, glyceryl trioctanoate, diisopropyl dimerate, synthetic lanolin derivatives and branched chain alcohols, sucrose esters of fatty acids, octyl hydroxystearate and mixtures thereof.

Preferably, the oils used may be selected such that the majority (at least about 75%, preferably at least about 80% and most preferably at least about 99%) of the types of oils used have solubility parameters that do not differ by more than from about 1 to about 0.1, preferably from about 0.8 to about 0.1.

A surfactant may also be added to compositions of the invention, in order to confer beneficial cosmetic or application properties. Surfactants suitable for use may be those which can form emulsions and/or association structures. Surfactant emulsifier can be from 0% to about 20% of the formulation, preferably from 0% to about 15% and most preferably from about 1% to about 10%. Examples of suitable emulsifiers can be found in U.S. Pat. No. 5,085,856 to Dunphy et al., and U.S. Pat. No. 5,688,831 to El-Nokaly et al. Examples of other suitable emulsifiers can be found in Cosmetic Bench Reference, pp. 1.22, 1.24-1.26 (1996), all of which are incorporated herein by reference.

Examples of surface active agents which may be used in the compositions of this invention include sodium alkyl sulfates, e.g., sodium lauryl sulfate and sodium myristyl sulfate, sodium N-acyl sarcosinates, e.g., sodium N-lauroyl sarcosinate and sodium N-myristoyl sarcosinate, sodium dodecylbenzenesulfonate, sodium hydrogenated coconut fatty acid monoglyceride sulfate, sodium lauryl sulfoacetate and N-acyl glutamates, e.g., N-palmitoyl glutamate, N-methylacyltaurin sodium salt, N-methylacylalanine sodium salt, sodium alpha-olefin sulfonate and sodium dioctylsulfosuccinate; N-alkylaminoglycerols, e.g., N-lauryl-diamino-ethylglycerol and N-myristyldiaminoethylglycerol, N-alkyl-N-carboxymethylammonium betaine and sodium 2-alkyl-1-hydroxyethylimidazoline betaine; polyoxyethylenealkyl ether, polyoxyethylenealkylaryl ether, polyoxyethylenelanolin alcohol, polyoxyethyleneglyceryl monoaliphatic acid ester, polyoxyethylenesorbitol aliphatic acid ester, polyoxyethylene aliphatic acid ester, higher aliphatic acid glycerol ester, sorbitan aliphatic acid ester, Pluronic type surface active agent, and polyoxyethylenesorbitan aliphatic acid esters such as polyoxyethylenesorbitan monooleate and polyoxyethylenesorbitan monolaurate. Emulsifier-type surfactants known to those of skill in the art should be used in the compositions of this invention.

Also useful herein may be surfactants that form association structures, preferably lamellar or hexagonal liquid crystals, at ambient temperature when mixed with a polar solvent. In preparing a sample combination of surfactant and polar solvent to demonstrate the ability to form association structures, the surfactant needs to be sufficiently soluble in the polar solvent such that an association structure can form at ambient temperature. One of ordinary skill in the art is capable of determining compatible interactions.

Any surfactant which forms association structures at ambient temperature and is suitable for use in cosmetics may be suitable for use herein. Surfactants suitable for use in cosmetics do not present dermatological or toxicological problems. Anionic surfactants, nonionic surfactants, cationic surfactants, amphoteric surfactants and mixtures thereof may be suitable for use. Preferably anionic surfactants, nonionic surfactants, cationic surfactants, amphoteric surfactants and mixtures thereof having a Krafft point at or below about ambient temperature are used. More preferably, nonionic surfactants, cationic surfactants, amphoteric surfactants and mixtures thereof having a Krafft point at or below about ambient temperature are used.

The surfactants can be used at levels from about 4% to about 97%, preferably from about 5% to about 95%, more preferably from about 20% to about 90% and most preferably from about 30% to about 70% of the association structure.

The cosmetic compositions of this invention may contain one or more materials, herein singly or collectively referred to as a “solidifying agent”, that are effective to solidify the particular liquid base materials to be used in a cosmetic composition. (As used herein, the term “solidify” refers to the physical and/or chemical alteration of the liquid base material so as to form a solid or semi-solid at ambient conditions, i.e., to form a final composition that has a stable physical structure and can be deposited on the skin under normal use conditions.) As is appreciated by those skilled in the art, the selection of the particular solidifying agent for use in the cosmetic compositions will depend upon the particular type of composition desired, i.e., gel or wax-based, the desired rheology, the liquid base material used and the other materials to be used in the composition. The solidifying agent can be preferably present at a concentration of from about 0% to about 90%, more preferably from about 1% to about 50%, even more preferably from about 5% to about 40%, most preferably from about 3% to about 20%.

The wax cosmetic stick embodiments of this invention preferably may contain from about 5% to about 50% (by weight) of a waxy solidifying agent. By the term “waxy solidifying agent,” as used herein, is meant a solidifying material having wax-like characteristics. Such waxy materials may also serve as emollients. Among the waxy materials useful herein are the high melting point waxes, i.e., having a melting point of from about 65° C. to about 125° C., such as beeswax, spermaceti, carnauba, bayberry, candelilla, montan, ozokerite, ceresin, paraffin, synthetic waxes such as Fisher-Tropsch waxes, microcrystalline wax, and mixtures thereof. Ceresin, ozokerite, white beeswax, synthetic waxes, and mixtures thereof, are among those useful herein; additional useful waxes are disclosed in U.S. Pat. No. 4,049,792, Elsnau, issued Sep. 20, 1977, herein incorporated by reference in its entirety. Low melting waxes, having a melting point of from about 37° C. to about 75° C., may be preferred for use in the wax stick embodiments of this invention. Wax stick embodiments of this invention, which contain volatile silicone oils as a liquid base material, preferably contain from about 10% to about 35%, more preferably from about 10% to about 20% (by weight), of a low-melting wax. Such materials include fatty acids, fatty alcohols, fatty acid esters and fatty acid amides, having fatty chains of from about 8 to about 30 carbon atoms, and mixtures thereof. Wax-like materials include cetyl alcohol, palmitic acid, stearyl alcohol, behenamide, sucrose esters of tallow fatty acids, mono and di-fatty acid esters of polyethylene glycol, and mixtures thereof. Stearyl alcohol, cetyl alcohol, and mixtures thereof, are mostly used. Additional fatty acids, fatty alcohols, and other wax-like materials useful in this invention are also well known in the art.

In addition, these compositions may include other medicinal agents, therapeutical agents, carriers, adjuvants, and the like. Some particular additional agents may include sunscreens; retinoids; antioxidants; hydroxyacids; fatty acids, acceptable non-toxic organic salts of metal derived from naturally occurring amino acids or from hydroxyalkyl acids; botanical extracts, salicylic acid, benzoyl peroxide, antibiotics, antiandrogens, anti-inflammatory agents, antioxidants, ascorbic acid, vitamins B, tocopherols or tocotrienols, corticosteroids, moisteners, surfactants, keratolytic agents, complexing agents, colorants, fragrances, and mixtures thereof.

### EXAMPLES

### Example 1

Clinical Evaluation for Dark Circles

Women subjects with mild to moderate dark circles under their edges are recruited for the study. Both an expert grader and the panelists evaluate the severity of the dark circles under their eyes prior to application of test products. The composition containing compounds of the invention is topically applied to the skin area around one eye and a composition not containing the inventive compounds around the opposite eye. Treatment assignments are randomized across the panel, and neither the panelist nor the grader, have knowledge of the treatment code. One hour after product application, both the grader and panelist can separately evaluate the appearance of the dark circles under the eyes.

### Example 2

Clinical Evaluation for Puffiness

A set of women subjects with puffiness under their eyes is recruited, and a composition containing the inventive compound is applied under one eye, and a composition with no inventive compound is applied under the other eye. The panelists use the product for 4 weeks, returning at week 2 for another dermatologist evaluation. After 2 and 4 weeks of product use, both the panelists and the dermatologist can evaluate the improvement in the puffiness of the eyes compared with the baseline observations.

### Example 3

Clinical Evaluation for Aging Signs

Expert graders that have been trained in visual and tactile evaluations assess the different aging signs of the face by grading on a semi-structured scale. Each subject is characterized by a quantitative profile of his or her aging signs and two expert graders evaluate each parameter at each time point.

Mean values and standard deviation are calculated, as well as variations of the parameter relative to before application (expressed in percentage). A Paired Student's t test is used to determine the significance of the results.

### Example 4

Measurements with Reviscometer® RVM 600

The Reviscometer® RVM 600, developed by Courage Khazaka Electronic GmbH, is an instrument used to measure skin firmness. This device consists of a probe that places two needle sensors on the skin. One sensor transmits an acoustical shockwave while the other receives the wave after it has propagated along the surface of the skin.

One determination is performed on each area and at each time point on the neck or arm. The place of the probe is marked with an ink, and a mask of the neck with ears, the spots and the most important wrinkles is done to reposition the probe exactly at the same place after one week of application and 45 minutes after the last application. Results are expressed for all the subjects at each time point.

The area under curve is considered and standard deviations are calculated. Paired Student's t-test is used to determine the significance of the results.

### Example 5

In Vitro Skin Model Test

The skin-lightening effect was validated on the in vitro skin model, the MelanoDerm™ carried out by MatTek using MEL-300B melanoderms. This model is very similar in structure and function to the natural skin and has the cell types relevant in the epidermis including the melanocytes, which are responsible for the synthesis of the main pigment melanin. Further information can be found, for example, in http://www.mattek.com/pages/in_vitro_basics. The study was carried out over a period of 14 days at two concentrations, 10 μM and 60 μM. 1% Kojic acid was the positive standard and untreated as negative control.

Digital image quantification was achieved by subtracting a standard background value from the pixel content of regions of identical size. The pixel content of untreated cells was set at 1.00. Treatment with topical 1% kojic acid as a positive control gave pixel density of 0.96 or 4% lightening. Treatment with Duroquinone gave pixel density of 0.82 or 18% lightening at a concentration of 10 μM and 0.60 or 40% lightening at a concentration of 60 μM. Treatment with 2,5,6-trimethyl-3-(4-(trifluoromethyl)phenyl)cyclohexa-2,5-diene-1,4-dione gave pixel density of 0.69 or 34% lightening at a concentration of 60 μM.

This demonstrates that Duroquinone is four and a half times as effective as the kojic acid used as positive standard at a concentration of 10 μM and ten times as effective at a concentration of 60 μM. 2,5,6-trimethyl-3-(4-(trifluoromethyl)phenyl)cyclohexa-2,5-diene-1,4-dione is ten times as effective at a concentration of 60 μM as kojic acid.

For all compositions described herein, and all methods using a composition described herein, the compositions can either comprise the listed components or steps, or can “consist essentially of” the listed components or steps. When a composition is described as “consisting essentially of” the listed components, the composition contains the components listed, and may contain other components which do not substantially affect the skin or the skin condition being treated, but do not contain any other components which substantially affect the skin or the skin condition being treated other than those components expressly listed; or, if the composition does contain extra components other than those listed which substantially affect the skin or the skin condition being treated, the composition does not contain a sufficient concentration or amount of the extra components to substantially affect the skin or the skin condition being treated. When a method is described as “consisting essentially of” the listed steps, the method contains the steps listed, and may contain other steps that do not substantially affect the skin or the skin condition being treated, but the method does not contain any other steps which substantially affect the skin or the skin condition being treated other than those steps expressly listed.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto. All patents and publications cited above are hereby incorporated by reference.

What is claimed is:

1. A cosmetic or dermatological composition comprising one or more benzoquinones of formula I or their reduced form of formula Ia: 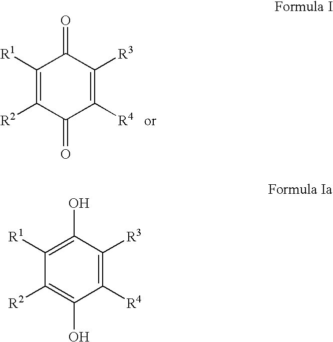
wherein R¹, R², R³, and R⁴ are independently selected from (C₁-C₆)alkyl, (C₁-C₆) arylalkyl or aryl, where the aryl group can be further substituted with (C₁-C₆)alkyl, (C₁-C₆)alkoxy, hydroxy, halogen or (C₁-C₆)haloalkyl,
and mixtures thereof.

2. The composition of claim 1, comprising a compound of formula I wherein R¹, R², and R³ are methyl, and R⁴ is phenyl optionally substituted with halogen or haloalkyl.

3. The composition of claim 1, comprising a compound of formula I wherein R¹, R², and R³ are methyl, and R⁴ is benzyl or phenylethyl optionally substituted with halogen or haloalkyl.

4. The composition of claim 1, comprising a compound of formula Ib wherein R¹, R², R³, and R⁴ are methyl, and the compound is Duroquinone (also named 2,3,5,6-tetramethylcyclohexa-2,5-diene-1,4-dione) or a compound of formula Ic, 2,3,5,6-tetramethylbenzene-1,4-diol, or a mixture thereof:

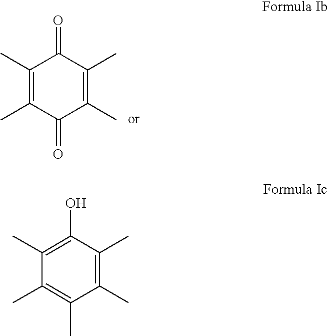

5. The composition of claim 4, comprising a compound of formula Ib.

6. A cosmetic or dermatological composition comprising a therapeutically effective amount of the composition of claim 1 and a pharmaceutically or cosmetically acceptable carrier.

7. A method of regulating or preventing a skin condition wherein the skin condition is characterized by oxidative stress or a degenerative process, comprising administering to a subject exhibiting said skin condition an effective amount of the composition of claim 1.

8. A method of reducing or treating the signs of skin aging or of reducing the appearance of signs of skin aging comprising administering to a subject exhibiting said skin condition an effective amount of the composition of claim 1.

9. A method of regulating or preventing a skin condition associated with visible and/or tactile discontinuities of the skin, comprising administering to a subject exhibiting said skin condition, an effective amount of the composition of claim 1.

10. A method of claim 9, wherein the visible and/or tactile discontinuities are associated with aging, age-related damage or damage resulting from extrinsic factors.

11. A method of claim 9, wherein the visible discontinuities of the skin are associated with pigmentation disorders.

12. A method of claim 11, wherein the pigmentation disorders are selected from uneven pigmentation, age spots, vitiligo and melasma.

13. A method for preventing, lightening or reducing the appearance of visible and/or tactile discontinuities of the skin, comprising administering to a subject exhibiting said skin conditions an effective amount of the composition of claim 1.

14. The method of claim 13, wherein the visible and/or tactile discontinuities of the skin are selected from the group consisting of increased pore size, flaking, mottling, wrinkles, furrows, and skin lines.

15. A method of regulating or preventing a skin condition wherein the skin condition is associated with visible and/or tactile discontinuities of the skin, while concurrently reducing or treating the signs of skin aging, comprising administering to a subject exhibiting said skin condition, an effective amount of the composition of claim 1.

16. The method of claim 15, wherein the visible discontinuities of the skin are selected from uneven pigmentation, age spots, vitiligo and melasma.

17. The method of claim 15, wherein the visible discontinuities of the skin are a result of harmful ultraviolet radiation, pollution or other environmental insults, stress or fatigue.

18. A method of preventing, lightening or reducing the appearance of visible and/or tactile discontinuities of the skin, while concurrently reducing or treating the signs of skin aging, comprising administering to a subject exhibiting said skin condition an effective amount of the composition of claim 1.

19. The method of claim 18, wherein the visible discontinuities of the skin are selected from uneven pigmentation, age spots, vitiligo and melasma.

20. The method of claim 19, wherein the visible discontinuities of the skin are a result of harmful ultraviolet radiation, pollution or other environmental insults, stress or fatigue

21. Use of a composition comprising a skin care composition of claim 1 in the manufacture of a cosmetic or dermatological composition for treating a mammalian subject of a dermatologic condition to prevent, regulate or treat signs of skin aging or skin pigmentation, or to reduce the appearance of skin aging or skin pigmentation.
